(19) United States
(12) Patent Application Publication
(10) Pub. No.: US 2008/0027523 A1
(43) Pub. Date: Jan. 31, 2008

(54) Cover for Cooling a Patient and Cooling Device Comprising Such a Cover

(75) Inventors: Wilhelm Behringer, Wien (AT); Fritz Sterz, Wien (AT); Rudolf Faworka, Wien (AT)

Correspondence Address: LERNER GREENBERG STEMER LLP, P O BOX 2480, HOLLYWOOD, FL, 33022-2480, US

(73) Assignee: EMCOOLS-EMERGENCY MEDICAL COOLING SYSTEMS AG, Vienna (AT)

(21) Appl. No.: 11/664,491

(22) PCT Filed: Aug. 11, 2005
(86) PCT No.: PCT/AT05/00324

§ 371 Date: Apr. 2, 2007

(30) Foreign Application Priority Data
Oct. 1, 2004 (AT) A 1643/2004

(51) Int. Cl. A61F 7/10; A61F 7/02

(52) U.S. Cl. 607/109; 607/111; 607/112

(57) ABSTRACT

A cooling cover for cooling at least a part of the body of a patient includes at least one cooling element containing a cooling fluid and is intended for placement on the body or body part. The cooling element is cooled to below the freezing point prior to its application. The cooling element contains a material that has good thermal conductivity as compared to the cooling fluid and is contained in the cooling element to absorb the cooling fluid.

The invention relates to a cover for cooling at least a part of the body of a patient, including at least one cooling element containing a cooling fluid and intended for placement on the body or body part, which cooling element is cooled to below the freezing point prior to its application.

Furthermore, the invention relates to a device for cooling at least a part of the body of a patient, including at least one above-described cooling cover and a cooling appliance.

The present invention is, in particular, related to the cooling of cardiac arrest or stroke patients. Nevertheless, its use is also possible with patients having suffered cerebral traumas, traumas of the spinal cord or septic shocks. Finally, the cooling cover according to the invention can be used for the cooling of injuries, sprains etc. Last, but not least, the cooling cover according to the invention can also be used to cool products such as, for instance, food products or the like.

Investigations have proved that the chances of survival of patients suffering from cardiac arrest can be substantially increased by reducing the body temperature after a successful resuscitation. The application of hypothermia not only reduces the oxygen consumption in the brain of a patient, but also decelerates various cellular decomposition processes which cause irrepairable neurological damage even after a successful restoration of the blood circulation. Despite advances in ambulatory and emergency care systems and the use of state-of-the-art medical technologies in intensive medicine, a patient's chances to survive cardiac arrest outside a hospital are still very small. The incidence of sudden cardiac arrest outside a hospital in industrial countries ranges between 36 and 128 per 100,000 inhabitants per year. If cardiac arrest victims do not receive treatment within the first 4 to 6 minutes, irreversible brain damage is likely to occur. The chances of survival will be reduced by 7 to 10% every minute without treatment. After 10 minutes only few reanimation attempts have been successful.

Current therapy after cardiac arrest concentrates on resuscitation efforts. The use of life-sustaining medical devices and highly advanced surgical techniques enable physicians to restore a victim's circulation even after an extended period of arrest, yet with the problem of irreversible brain damage. Even after the restoration of the spontaneous circulation, the fatal brain and organ damaging process will continue due to chemical and physical blood changes during cardiac arrest (post-reanimation syndrome). The pathophysiologic mechanisms responsible for brain damage before, during and after reanimation are manifold. So far, no specific therapy has been provided to protect the brain after the restoration of the spontaneous circulation. Biomedical pharmaceuticals that are able to inhibit the mechanisms of cellular destruction have constituted a highly promising field of research, albeit still in the early stages. Research groups throughout the world have, therefore, been investigating other options to cope with those fatal mechanisms.

At present, hypothermia is the most advanced medical concept for the prevention or alleviation of post-reanimation syndrome. Numerous studies have proved the marked positive effect of hypothermia after specific ischemic conditions and, in particular, cardiac arrest. Unlike uncontrolled hypothermia, therapeutic hypothermia as used for cardiac or neurosurgery or for reanimation after cardiac arrest requires controlled conditions. Therapeutic hypothermia defines different grades of cooling:
Mild hypothermia: 36-33° C.
Moderate hypothermia: 32-28° C.
Deep hypothermia: 27-11° C.
Profound hypothermia: 10-6° C.
Ultraprofound hypothermia: 5-0° C.

Studies investigating the application of mild hypothermia as compared to normothermia in comatose survivors of a cardio-logically caused cardiac arrest have demonstrated that a lowering of the body temperature improves the survival rate and neurological recovery in such patients. In July 2003, the American Heart Association issued the recommendation to cool the victims of cardiac arrest outside a hospital by mild hypothermia. This recommendation was already provided in Europe in October 2002 by ILOR (International Liaison Committee of Resuscitation), to which ERC (European Resuscitation Council), AHA (American Heart Association) and many other worldwide associations belong and which has endeavoured to develop uniform guidelines for cardiopulmonal reanimation (CPR).

With mild hypothermia, the time of onset of cooling and its duration are of decisive importance. Presently available cooling methods are not suitable for an early induction of hypothermia. Although an immersion into ice water causes relatively rapid cooling, it is virtually inoperable. Cooling occurs too slow during the removal of clothes and the application of ice packages on the head and torso. The extracorporal cooling of blood is the fastest method for reducing the temperature, yet it involves logistic problems. Although the use of a cardiopulmonal bypass and a heat exchanger result in a rapid temperature reduction, cooling will be delayed by the time that is necessary to obtain vascular accession and prepare the devices. Even the intravenous infusion of large volumes of icecold fluid will only result in a slow cooling of the patient.

Mild hypothermia should be initiated as rapidly as possible after successful resuscitation. As opposed to mild hypothermia after successful resuscitation, another use of hypothermia turned out to be highly promising in an animal experiment, namely the very rapid induction of deep hypothermia already during cardiac arrest for the subsequent transport of the patient to the hospital under the protection of cooling and resuscitation of the same only at the hospital under controlled conditions (suspended animation). However, this concept still has to be proved in animal experiments.

Rapidly induced hypothermic cooling to prevent the mechanism of cellular destruction is not limited to cardiac arrest victims. Other possible indications at which a lowering of the body temperature has proved to be beneficial include cardiac infarction, apoplexy, brain trauma, spinal cord injuries or septic shock.

Currently available non-invasive cooling devices are unable to rapidly cool a patient, since the low temperature has to be transported through the skin and muscles and those systems act only partially and not over the entire body surface. Existing devices are, moreover, very large, heavy and difficult to handle and require relatively long preparation work. In addition, the available devices will usually need continuous electric supply, which will not be available, for instance, in ambulances.

US 2002/0193852 A1 describes a light-weight, portable system for warming or cooling a patient, which includes a device for providing a liquid cooling medium and a device through which the cooling medium is circulated for delivering to the patient the cold transported by the cooling medium. The delivery device is arranged to enclose the patient, leaving free the patient's face. The bag-like delivery device contains spacers between which cavities are formed for the guidance of the cooling medium. The cooled or heated liquid is introduced on the end of the bag and carried off on the opposite end. Apart from the involved tightness problems, the described apparatus is very bulky and heavy because of the large amounts of liquid required. Moreover, only relatively low cooling rates will be achieved by this method. Finally, the patient's head is only insufficiently covered by the cooling liquid and, hence, insufficiently cooled. Furthermore, the enclosure of the patient would not allow the realization of an examination or therapy such as, e.g., a cardiac massage on the patient.

Other known cooling devices involve the drawback of frequently cooling the skin to temperatures of below 0° C., thus causing burns on the skin. The cooling of body parts by the aid of, for instance, ice cubes or cooling bags containing coolants having freezing points of below 0° C. (such as, e.g., frozen salts, alcoholic solutions or gases), which are stored in deep-freezers, is dangerous, because the direct application of such a cooling element will cool the skin to temperatures of below 0° C., which may lead to injuries. Cooling with, for instance, ice cubes involves the problem of an insulation layer of water forming between the body surface and the ice cube because of the melting ice. Due to the poor thermal conductivity of water, optimal body cooling is not possible.

Departing from this prior art, the present invention is based on the object to provide a cover for cooling at least a part of the body of a patient suffering, in particular, from cardiac arrest, of the above-defined kind, by which cooling rates as high as possible will be obtained without doing any harm to the patient by too low temperatures. The cooling cover is to be as small and light-weight as possible so as to allow its use even outside hospitals, for instance in ambulances but also outside such facilities. The cooling cover is to be applicable without the demand for specially trained personnel. In addition, the cooling cover is to be producible as cost-effectively as possible so as to enable its use as a disposable product.

A further object of the present invention resides in providing an above-defined device for cooling at least a part of the body of a patient, including at least one above-defined cooling cover and a cooling appliance, by which cooling rates as high as possible are obtained and which is designed as small and light-weight as possible. The cooling device is to be applicable as independently as possible of external power supplies so as to enable its use even outside hospitals or ambulances.

The drawbacks of known systems are to be avoided or reduced.

The first object according to the invention is achieved in that a material having a good thermal conductivity as compared to the cooling fluid is contained in the cooling element to absorb the cooling fluid. This characteristic feature ensures that the usually poor thermal conductivity of the cooling fluid, for instance water, will be bridged and the melting temperature of the cooling fluid will be reached very quickly upon application of the cooling cover on the patient's skin due to the good thermal conductivity. The large melting heat of the ice can, thus, be used for cooling purposes. Provided the appropriate cooling fluids are chosen, frostbites on the skin will, thus, be avoided. If an appropriate heat capacity is generated by the cooling cover, particularly rapid cooling of the body merely by the application of the cooling cover will be ensured. By the combined use according to the invention, of a cooling fluid, in particular water, contained in a material exhibiting a comparatively good thermal conductivity, it will be feasible to reach the high cooling rates desired. To this end, it is necessary that the heat capacity of the cooling cover be accordingly large in order to ensure the cooling of a patient's body or body part. In doing so, the melting heat of ice, i.e. the heat absorbed by ice to become liquid, is utilized to cool the body. The material having a good thermal conductivity inhibits the formation of an insulating water layer which would prevent further cooling of the body or body parts of the patient. An advantage over other known systems is that the application of the cooling cover is particularly simple and can, thus, also be performed by untrained personnel, and that the cover can, moreover, also be briefly lifted to carry out an examination or therapy (such as, e.g., cardiac massage). Finally, false indications are rather unlikely with the cooling cover according to the invention, since no damage to the skin and less damage to the inner organs will be caused by the cooling cover on account of the combination according to the invention, of a good thermal conductivity and high heat capacity. The cooling cover can vary in size and thickness as a function of its application.

The thermally well conductive material may be comprised of a metal wool which is made of a metal or metal alloy having a good thermal conductivity, e.g. aluminum, copper or steel. The metal wool in every cooling element is enclosed by an appropriate sheath and soaked with the cooling fluid. Upon cooling of the cooling cover, for instance in a freezer, the liquid cooling medium penetrating the metal wool has assumed a solid state. When applying the cooling cover, the inherently poor thermal conductivity of the cooling fluid is enhanced by the metal wool so as to ensure a rapid heat or cold transfer from the cooling cover to the body and, hence, a rapid decrease of the temperature on the skin surface to the melting temperature of the cooling liquid. If the melting temperature of the cooling liquid is not substantially below 0° C., no burns need be feared on the skin.

It is also possible to form the thermally well conductive material by a metal foam made of a metal or metal alloy having a high thermal conductivity, e.g. aluminum, copper or steel. Metal foam is a material made of a metal and having a particularly low weight and high mechanical stability. In addition, the pores contained in the metal foam enable its permeability to a fluid and provide a large inner surface area.

The metal foam is preferably an open-pore foam so as to enable the absorption of as much cooling fluid as possible.

It is further possible to use graphite as the thermally well conductive material. Graphite has a higher thermal conductivity, and is also lighter, than the above-mentioned metals. Further-more, this material is also cheaper and biologically harmless. Graphite may also be used in the form of so-called expanded graphite. Graphite has a huge liquid absorption power. Volumes filled with graphite can, for instance, be filled with water by up to 90%. This material is, thus, excellently suitable for an application according to the invention.

In order to safely avoid injuries to the patient's skin by too low temperatures, the cooling fluid is comprised of water. Since water has a melting point of 0° C., no temperatures of below 0° C. will occur on the skin and, hence, no burns of the skin will be caused. In a preferred manner, super-clean water is used. Also is the melting heat of water relatively high with 335 kJ/kg. Melting heat is the heat that is absorbed by ice to become liquid.

In order to obtain a cooling cover that is flexible in its application, several cooling elements are advantageously arranged on a flexible support. Provided suitable dimensions are chosen for the cooling elements, the optimal adaptation of the cooling cover to the different surfaces of the body parts to be cooled will be feasible.

The flexible support is preferably made of latex. This material, which is made of natural rubber, is particularly easy to process, relatively inexpensive and highly extensible. In addition, this material is environmentally safe and decayable and withstands the low temperatures involved.

The flexible support may also be made of silicone. This material is particularly flexible and extensible such that the cooling cover can easily be placed on the skin.

According to a further characteristic feature of the invention, a heat-insulating layer is provided for arrangement between the cooling elements, or the support, and the body or body part. This will provide a better protection of the skin surface against hypothermy, what may be suitable in various applications. Naturally, the heat-insulating layer can be directly fixed to the cooling cover or produced in one piece with the same.

In order to prevent the support from tearing, it may include a reinforcement layer formed, for instance, by a fabric.

In order to obtain as high a heat capacity of the cooling element as possible while, at the same time, enabling small dimensions of the latter, especially in terms of height, the at least one cooling element is preferably designed with a parallelepipedic shape.

In order to prevent the cooling cover from rapidly warming due to the ambient air, a heat insulation may be arranged on the side facing away from the body, of the at least one cooling element. Such in insulation can be obtained by various materials with poor thermal conductivities, which are easily workable.

In addition, a reflection layer may be arranged on the side facing away from the body, of the at least one cooling element so as to avoid or reduce warming of the cooling cover, for instance by solar radiation.

Like the support, the cooling element may be made of latex. As already pointed out above, this material is particularly easy to process, relatively inexpensive and highly extensible.

The cooling element may also be made of silicone. As already pointed out above, this material is particularly flexible and extensible.

A plate made of a material having a particularly high thermal conductivity may be arranged on the surface facing the body, of the at least one cooling element so as to promote the transfer of cold to the patient. The plate may be a metal plate.

Alternatively, the support may be designed to have a reduced thickness, at least on the sites below the cooling elements, in order to ensure an optimum heat transfer.

On the at least one cooling element or on the support means for connection with other cooling elements or supports, respectively, may be provided to obtain a flexible configuration of the cooling cover. This enables several cooling elements to be modularly arranged one beside the other and connected with one another. The size and shape of the resulting cooling cover is adapted to the respective case of use.

The connection means may be formed by zippers.

In order to prevent the cooling cover from slipping on the patient, a means for fixing to the patient, for instance a belt with a quick-lock closure such as a Velcro closure, may be provided on the at least one cooling element or on the support. If this fixing means is directly fastened to the cooling cover, it will be ensured that the fixing means will be at hand in the case of use. This is of particular importance for the application in cardiac arrest patients, since in those cases rescue measures have to be taken particularly quickly.

For a better contact of the cooling cover on the patient, an adhesive layer may also be provided on the cooling element, on its surface facing the body. Prior to the application of the cooling cover, a cover foil attached to the adhesive layer is preferably pulled off, whereupon the cooling cover will adhere to the patient's skin. In this case, skin-safe adhesives are preferably used. The adhesive layer can be applied to at least parts of the lower side of the cooling cover, for instance in the liquid state, and subsequently covered by a suitable foil. It is likewise possible to attach the adhesive layer in the form of double-sided adhesive tapes to the cooling cover side facing the body.

If incisions, perforations or the like are provided between the cooling elements, the separation of the cooling cover, and its adaptation to the respective requirements in terms of size, will be particularly easy and preferably feasible without using a tool.

According to a further characteristic feature of the invention, sensors for measuring the temperature of the patient may be provided. Such sensors, which may also be connected with the appropriate electronics and with acoustic or visual output means, will, for instance, allow the monitoring of the temperatures prevailing on the surface of the skin so as to take the appropriate measures on grounds of the detected temperature values. Temperature monitoring also in the core-near region of the body is of special importance, because, for instance, cooling of the cardiac muscle to below 30° C. will again involve the risk of cardiac arrest.

If the cooling cover is used for cardiac arrest patients, it will be advantageous if an electric device for cardiac massage is provided. In this manner, the cooling of a patient can be combined with a simultaneous, automatic cardiac massage. Automatic pumps for cardiac massage are buckled around the patient's thorax. Periodic pressure pulses acting on the thorax will maintain the blood circulation. If such an automatic cardiac massage device is combined with the cooling cover according to the invention, the chances of survival of a patient will even further increase.

Depending on the application, the cooling elements can be provided in the form of a blanket or a sleeping bag.

For cooling the brain, the form of a headgear may be chosen as well. In this case, the size of the cooling elements must, of course, be adapted accordingly. For a headgear, smaller cooling elements than for a blanket would be employed to obtain smaller bending radii of the cooling cover.

It is likewise possible to arrange the cooling elements in the form of a flexible tube for covering a patient's arm or leg.

Furthermore, the cooling elements may be arranged in the form of a mitten or stocking, for instance in the event of a sprain of the hand or foot.

When using different cooling fluids or even differently sized cooling elements, a code, preferably a color code, may be provided to assist the selection of the respective cooling cover. This will, for instance, enable the physician or ambulanceman to quickly select and apply the appropriate cooling cover.

The second object according to the invention is achieved by an above-defined device, wherein the cooling appliance is designed to cool the cooling cover to a temperature of below 0° C. In doing so, it is merely decisive to freeze the cooling fluid in order to utilize, for the cooling procedure, the melting heat that is being absorbed as the cooling fluid passes from the frozen into the liquid state. Cooling to further below the freezing point will hardly improve the overall balance.

The cooling appliance may be comprised of an electrically operated refrigerating unit in the manner of a deep-freezer.

The cooling appliance may likewise be comprised of a Peltier element.

For ambulances or other cases of application, it may be advantageous that the cooling appliance does not need any external power source and is merely comprised of a container with a heat insulation to receive the cooling cover. The cooling cover is first of all cooled in a deep-freezer before it is stored over a predetermined time in the above-mentioned passive container including the heat insulation. By selecting the appropriate heat insulation, it will be feasible without external power supply to reach a period of from some days up to a week during which no warming of the cooling cover will occur, what would render its further application impossible.

A heat insulation made of evacuated silicic acid will be particularly efficient for the container. Heat insulation foils made of silicic acid and sold by Wacker under the name of WDS®, for instance, exhibit excellent insulating properties.

For the application in emergency vehicles and also in out-patient's clinic departments, it will be advantageous if the cooling appliance is integrated in a patient gurney. The cooling cover will, thus, be ready to hand at any time and can be quickly applied so as to ensure a higher chance of survival with cardiac arrest patients.

According to a further characteristic feature of the invention, at least one sensor for measuring the temperature is provided. Such a sensor, which may be connected with an evaluation unit and, optionally, with an acoustic and/or visual output unit, allows for the documentation of the temperature prevailing, for instance, in the cooling appliance so as to enable special measures to be taken if, for instance, a predetermined temperature is being exceeded.

It should finally be mentioned that the cooling cover and cooling appliance according to the invention cannot only be used in men, but theoretically also with animals.

The invention will be explained in more detail by way of the accompanying drawings. Therein:

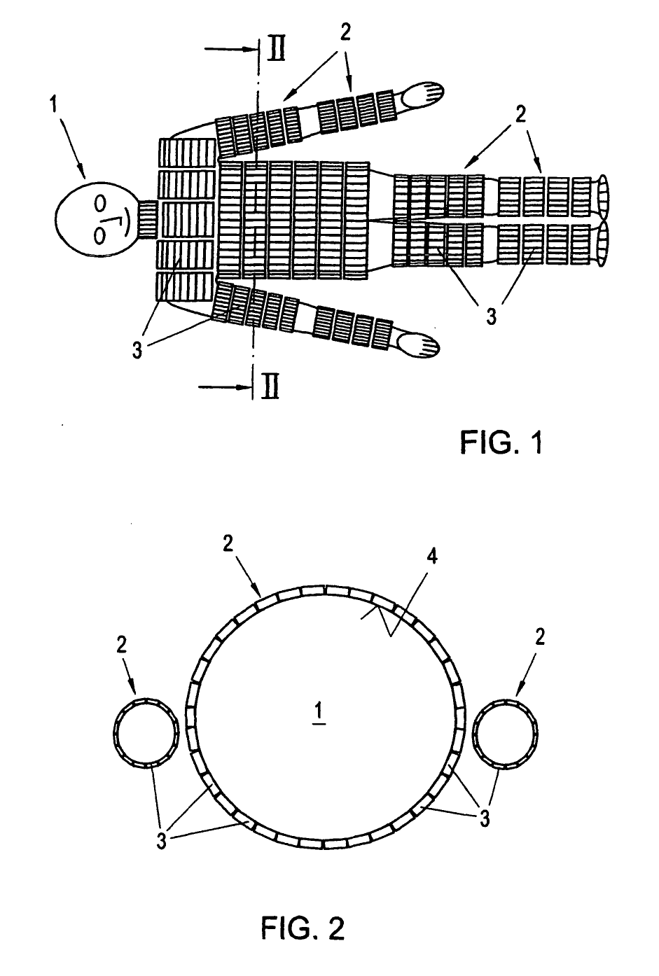

FIG. 1 is a schematic view of a patient with cooling covers applied thereon;

FIG. 2 illustrates a section through the patient along the sectional line II-II of FIG. 1;

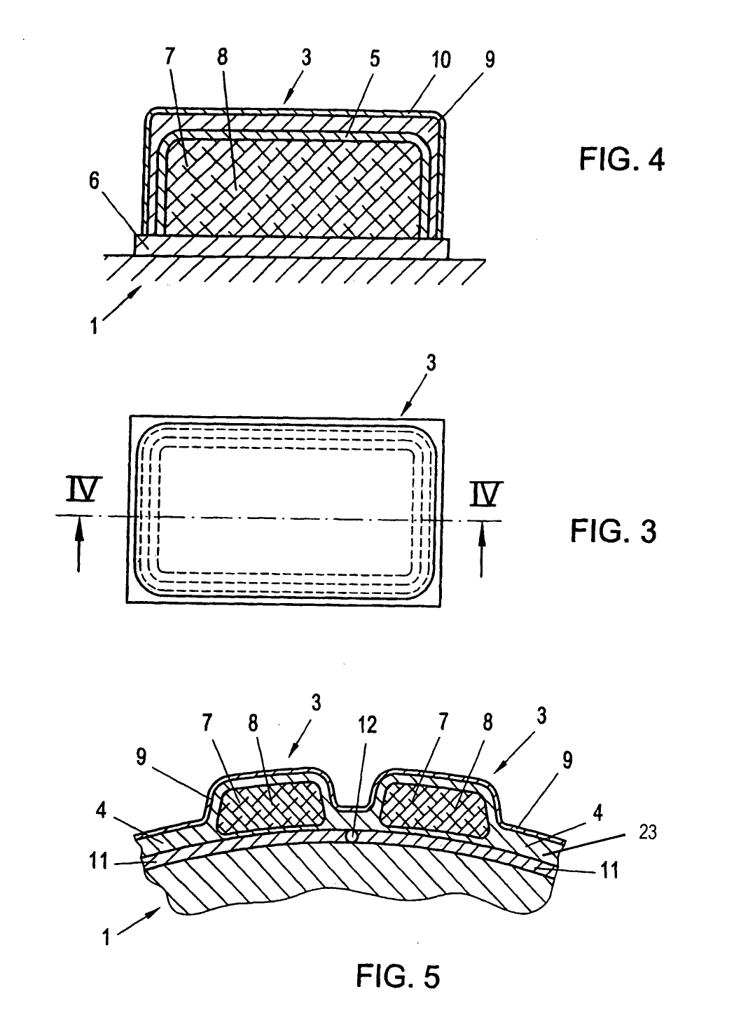

FIG. 3 is a top view on a cooling element of a cooling cover;

FIG. 4 illustrates a section along sectional line IV-IV through the cooling element according to FIG. 3;

FIG. 5 illustrates a section through a part of a cooling cover on an enlarged scale;

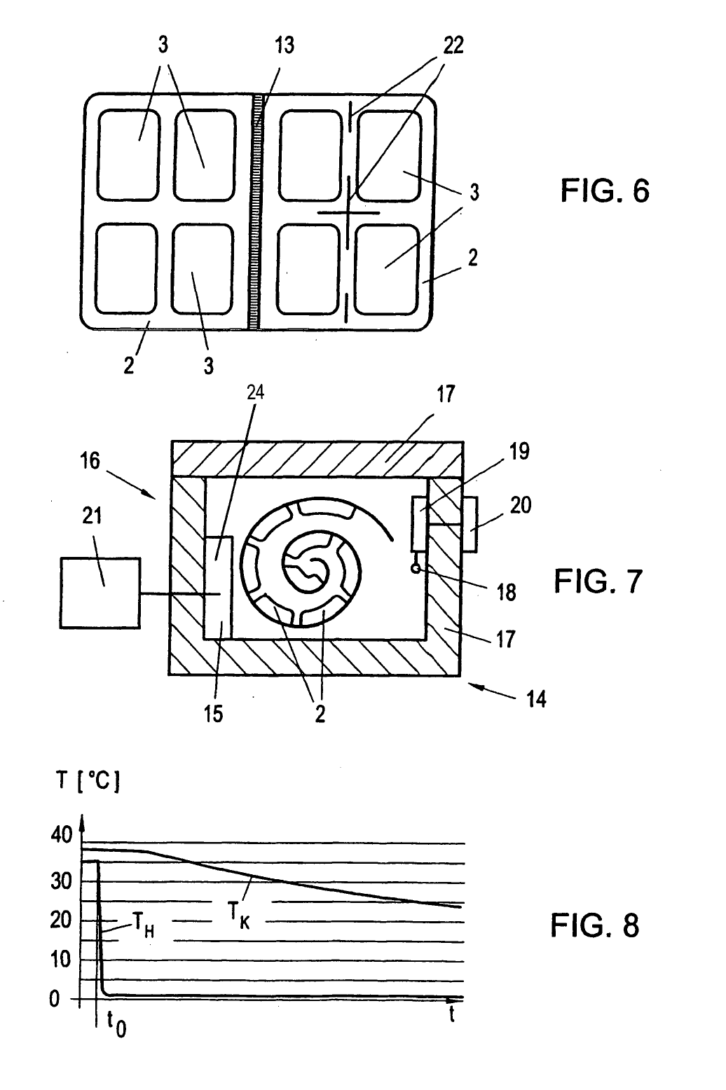

FIG. 6 is a top view on a cooling cover assembled of several elements;

FIG. 7 illustrates a section through a cooling device for cooling a cooling cover; and

FIG. 8 indicates the temperature courses on the skin and below the skin in an animal experiment.

FIG. 1 is a schematic top view on a patient 1, on whom cooling covers 2 according to the invention are arranged both on the upper body and on the extremities. The cooling covers 2 are each comprised of at least one cooling element 3, which will be explained in more detail below. Depending on the case of application, the cooling covers 2 may be designed to be planar or hose-shaped. The cooling covers 2 are particularly quick and simple to apply and, due to the characteristic features according to the invention, will prevent the skin from cooling to too low a temperature and, hence, the formation of burns. On the other hand, the cooling covers 2 enable a rapid lowering of the body temperature and, for instance in the case of cardiac arrest, an increase in the chances of survival and the chances of complete recovery.

FIG. 2 is a section, along sectional line II-II, through the patient 1 according to FIG. 1. Hose-shaped cooling covers 2 are arranged about the thorax and the arms. For an easier application of the cooling covers 2, the latter may be designed to be planar so as to be laid and fixed around the body or body part of the patient 1. With cardiac arrest patients, it is essential to cover with the cooling covers 2 the breast region, the back region for the protection of the medulla, and the head region for the protection of the brain. The cooling covers 2 are preferably comprised of several cooling elements 3 which are arranged on a flexible support 4 made, for instance, of latex. Instead of using a support 4 the cooling elements 3 may, of course, also be connected with one another.

FIG. 3 is a top view on a cooling element 3 of, for instance, parallelepipedic shape. As is apparent from the sectional view of FIG. 4, the cooling element 3 is comprised of an enclosure 5 made of a cold-resistant, extensible synthetic material such as, e.g., latex or silicone. The enclosure 5 is connected with a contact plate 6, which is preferably made of a thermally conductive material such as, e.g., metal or a thermally conductive synthetic material. Naturally, the enclosure 5 and the contact plate 6 may also be made in one piece. In this respect, latex is particularly suitable, because it is easily workable. In addition, this material is environmentally safe and withstands low temperatures without deterioration of its properties. The cooling element 3 contains a thermally well conductive material 7 in which the cooling fluid 8 is embedded. On account of the thermally well conductive material 7, which may, for instance, be comprised of metal wool, metal foam or graphite, the thermal conductivity is enhanced and the cold of the cooling fluid 8 will, thus, be more rapidly transported to the surface of the body of the patient 1. In order to prevent or reduce the external warming of the cooling fluid 8 contained in the cooling element 3, a heat insulation 9 may be arranged on the side of the cooling element 3 facing away from the body of the patient 1. In addition, a reflection layer 10 may also be provided on the heat insulation 9 to prevent warming, for instance, by solar radiation. This reflection layer 10 can, for instance, be produced by applying a mixture of latex with aluminum particles to be simply sprayed on the cooling cover 2. The cooling element 3, or an array of several cooling elements 3 provided on a support 4, is placed on the respective body region of the patient 1. The good thermal conductivity of the material 8 contained in the cooling element 3 causes the rapid cooling of the skin surface of the patient 1 and, hence, also the relatively rapid lowering of the core temperature of the patient 1.

FIG. 5 is a sectional view through a part of a cooling cover 2 in which the cooling elements 3 are arranged on a flexible support 4. Here, the cooling elements 3 are not arranged in a parallelepipedic manner but in the form of truncated pyramids, which provides easier producibility and enhanced stability. The cooling elements 3 can also be formed in one piece with the flexible support 4. The thermally well conductive material 8 and the cooling fluid 7 are contained in the interior of the cooling elements 3. In order to enhance the heat transfer from the patient 1 to the cooling element 3, the support 4 is preferably designed to have a reduced thickness in the region of the cooling elements 3 as compared to the remaining regions. It is, of course, likewise possible to arrange a contact plate 6 on the side of the cooling elements 3 facing the body of the patient 1 (cf. FIG. 4). For certain applications, a heat-insulating layer 11 can be arranged between the cooling cover 2 and the skin surface of the patient 1 in order to prevent too rapid under-cooling of the skin of the patient 1 to below predetermined temperature values. To monitor the temperature on the skin surface of the patient 1, a sensor 12 may be provided, which is either loosely placed or glued on the skin of the patient 1 or arranged in the heat-insulating layer 11 or in the support 4 of the cooling cover 2. The temperature sensor 12 is connected with a suitable evaluation electronics and, optionally, an acoustic or visual output unit in order to indicate to the physician or ambulanceman the respective temperature prevailing on the skin. As already mentioned above, at least parts of the side of the cooling cover 2 facing the body of the patient 1 may be provided with an adhesive layer (not illustrated) to provide a better connection with the skin surface of the patient 1.

FIG. 6 is a top view on two cooling covers 2 which are each comprised of four cooling elements 3 equipped with connection means 13 such as, for instance, zippers. In this manner, a suitable cooling cover 2 can be formed of several modules. Between the cooling elements 3, the cooling cover 2 may also be provided with incisions 22, perforations or the like. These serve to prevent the formation of an insulating air cushion between the skin surface of the patient 1 and the cooling cover 2 and, on the other hand, enhance the flexibility of the cooling cover 2. The incisions 22 can be simply and quickly produced by punching, for instance after the manufacture of the cooling cover 2. Besides, the cooling cover 2 can be more easily separated in the region of such incisions 22 or perforations, preferably without using a tool, in order to adapt the cooling cover 2 to the respective conditions in terms of size.

A combination of the cooling cover 2 with an automatic heart massage device (not illustrated) is optimal, too.

FIG. 7 is a sectional view through a cooling appliance 14 for cooling the described cooling covers 2 or for the protection of already cooled cooling covers 2 against warming. The cooling appliance 14 is preferably designed to cool the cooling cover 2 to temperatures of below 0° C., or below the freezing point of the cooling fluid 8 and comprises a cooling aggregate 15 to be connected with an electric supply 16. The cooling appliance 14 may also be formed by a passive container 21 with a heat insulation 17 to receive the cooling cover 2. When selecting the appropriate heat insulation 17, an already cooled cooling cover 2 can be stored for several days without power supply. In the container 16 of the cooling appliance 14, a sensor 18 for measuring the temperature may be provided, which may be connected with an evaluation unit 19 and, optionally, an acoustic or visual output unit 20. The readiness for use of the cooling cover 2 can, thus, be monitored.

An application in which the cooling appliance 14 is integrated in a patient gurney is of particular interest. This enables an especially quick use of the cooling covers 2, which is of particular importance in the event of a cardiac arrest of the patient 1 (not illustrated).

FIG. 8 finally shows the course of the temperature T_{H} on the skin surface, and the body temperature T_{K} in a depth of 27.5 mm below the skin, of an experimental animal when applying a cooling cover 2 according to the invention in an animal experiment. Pigs each having a weight of 75-95 kg were provided with cooling covers 2 according to the invention. The cooling elements 3 contained pure water embedded in aluminum chips. At time t₀, the cooling cover is placed on the experimental animal, whereupon the skin temperature T_{H} drops to 0° C. within a few seconds. Further lowering of the temperature to below 0° C. is impossible because of the use of water as a cooling fluid 7. Thus, no frostbites can occur on the skin of the experimental animal. The body temperature T_{K} starts to drop already some minutes after the application of the cooling cover 2 at time to, finally reaching 32-33° C. after approximately 15 minutes. The body temperature T_{K} continues to drop as a function of the duration of application and reaches 24-25° C. after approximately 30 minutes. The time history of the body temperature T_{K} depends on the circulation of the experimental animal or patient 1 and on the size of the cooling cover 2. In the animal experiments using pigs, a lowering of the brain temperature of 5° C. was obtained within approximately 30 minutes. Approximately 0.6 m² was covered by the cooling cover 2.

The cooling device 1 according to the invention enables the particularly rapid cooling of patients, in particular cardiac arrest patients, even outside hospitals or similar institutions so as to increase the chance of survival and reduce the risk of cerebral damage. The device can also be applied to other cases where mild or more intense hypothermia will be beneficial.

The invention will be explained in even more detail by way of an example. The Table below indicates for some materials the values or value ranges for the specific heat capacity c, the thermal conductivity λ and the density ρ.

| Heat capacity c | | Thermal conductivity λ | Density (Weight) ρ |
|---|---|---|---|
| KJ/kg ° C. | | W/m · K | g/cm³ |
| Aluminum | 0.9 | 230 | 2.71 |
| Graphite | 0.7 | 170 to 370 | 2.2 |
| Copper | 0.38 | 390 | 8.97 |
| Water | 4.186 | 0.57 | 1 |
| Ice | 2.1 | 1.7 | |
| Muscle tissue | 3.6 | 0.36 to 0.5 | 1 |
| Bones | 1.2 | 0.5 | 2 |
| Fat | 1.67 | 0.186 to 0.3 | 0.93 |
| Blood | 4 | 0.472 to 0.62 | 1 |

Aluminum and graphite have approximately identical properties in terms of thermal conductivity λ. In terms of weight and volume, based on the specific heat capacity c, graphite offers advantages over aluminum. Water has a very poor thermal conductivity λ. If water is, for instance, supplemented with 10 vol. % aluminum or graphite, its thermal conductivity λ will increase by approximately 20 times. By introducing a cooling fluid, in particular water, into a material that has a very good thermal conductivity X as compared to water, the poor thermal conductivity of the latter will be bridged. The heat capacity c of ice will not be substantially influenced by the relatively small volume of aluminum, graphite or copper. The heat capacity c of ice, thus, combines with the thermal conductivity λ of aluminum, graphite, copper or the like. By freezing the water to −5° C. to −20° C., a heat absorbability of about 10-40 kJ/kg will be provided in order to reach the desired temperature of 0° C. on the skin surface.

According to a very rough presumption, a specific heat capacity c of 4 kJ/kg.° C. can be anticipated for human tissue. At a skin temperature of 35° C., a heat absorbability of 140 kJ/kg, i.e. a heat absorbability that is 3 to 14 times larger than that of the cooling mat, will result. It is, thus, impossible for the cooling cover to cause frostbites on the skin. During the melting of ice, the cooling effect occurs through the melting heat of ice. However, while normal ice would cause a water layer to form between the skin surface and the ice, which would prevent further cooling of the body, the present invention inhibits the formation of an insulating layer and, hence, ensures effective cooling.

To cool a human body having a weight of about 90 kg and a body temperature of 37° C. by 5° C., a heat amount Q=c.m.ΔT=4.90.5=1800 kJ will be required. To this end, a mass of slightly more than 5 kg ice will be required. There was a high consistency between the theoretical values and the practical values tested in animal experiments. In the studies on pigs having weights of 75 to 95 kg, seven cooling covers of 14 cm×38 cm and seven mat pieces of 8 cm×30 cm each and a head hood of 15 cm×40 cm were each applied. This yields a surface of approximately 0.6 m². The cooling covers were frozen at −15° C. The obtained temperature drops by 5° C. in the brains of the pigs occurred within 30 minutes.

The concrete practical results have to be substantiated and optimized by further experiments.

As already pointed out above, the cooling cover may also be used to cool products such as, for instance, food products or the like.

1-41. (canceled)

42. A cooling cover for cooling at least a part of a body of a patient, the cover comprising: at least one cooling element containing a cooling fluid and intended for placement on one of the body and a body part, said cooling element being cooled to below a freezing point prior to its application, said cooling element containing a material having good thermal conductivity as compared to said cooling fluid and absorbing said cooling fluid.

43. The cooling cover according to claim 42, wherein said material is formed of a metal wool made of a substance selected from the group consisting of metal and metal alloy having a good thermal conductivity.

44. The cooling cover according to claim 42, wherein said material is formed of a metal foam made of a substance selected from the group consisting of metal and metal alloy having a high thermal conductivity.

45. The cooling cover according to claim 44, wherein said metal foam is an open-pore foam.

46. The cooling cover according to claim 42, wherein said material is graphite.

47. The cooling cover according to claim 42, wherein said cooling fluid is water.

48. The cooling cover according to claim 42, further comprising a flexible support and said at least one cooling element is one of several cooling elements disposed on said flexible support.

49. The cooling cover according to claim 48, wherein said flexible support is made of silicone.

50. The cooling cover according to claim 49, wherein said flexible support is made of latex.

51. The cooling cover according to claim 42, further comprising:
a flexible support supporting said at least one cooling element; and
a heat-insulating layer disposed between said flexible support and one of the body part and the body.

52. The cooling cover according to claim 48, wherein said flexible support has a reinforcement layer.

53. The cooling cover according to claim 42, wherein said at least one cooling element has a substantially parallelepipedal shape.

54. The cooling cover according to claim 42, further comprising a heat insulation layer disposed on a side of said at least one cooling element facing away from the body.

55. The cooling cover according to claim 42, further comprising a reflection layer disposed on a side of said at least one cooling element facing away from the body.

56. The cooling cover according to claim 42, wherein said at least one cooling element is made from a material selected from the group consisting of latex and silicone.

57. The cooling cover according to claim 42, wherein said at least one cooling element is one of a plurality of cooling elements made from a material selected from the group consisting of latex and silicone.

58. The cooling cover according to claim 42, further comprising a contact plate made of a further material having a particularly high thermal conductivity and disposed on a side of said at least one cooling element facing the body.

59. The cooling cover according to claim 48, wherein said flexible support has an area of a reduced thickness, at least below said at least one cooling elements, as compared to remaining regions of said flexible support.

60. The cooling cover according to according to claim 48, further comprising connection means disposed on said at least one cooling element and said flexible support for connecting with other cooling elements or other flexible supports.

61. The cooling cover according to claim 60, wherein said connection means are zippers.

62. The cooling cover according to claim 48, further comprising a fixing means for fixing to the patient and disposed on one of said at least one cooling element and said flexible support.

63. The cooling cover according to claim 62, wherein said fixing means is a belt.

64. The cooling cover according to claim 42, further comprising an adhesive layer disposed on a side of said at least one cooling element facing the patient.

65. The cooling cover according to claim 42, wherein said cooling element is one of plurality of cooling elements and the cooling cover has one of incisions and perforations formed therein between said cooling elements.

66. The cooling cover according to claim 42, further comprising sensors for measuring a temperature of the patient.

67. The cooling cover according to claim 42, wherein said at least one cooling element is one of a plurality of cooling elements and said cooling elements form a blanket.

68. The cooling cover according to claim 42, wherein said at least one cooling element is one of a plurality of cooling elements and said cooling elements form a sleeping bag.

69. The cooling cover according to claim 42, wherein said at least one cooling element is one of a plurality of cooling elements and said cooling elements form headgear.

70. The cooling cover according to claim 42, wherein said at least one cooling element is one of a plurality of cooling elements and said cooling elements form of a flexible tube for receiving an arm or leg.

71. The cooling cover according to claim 42, wherein said at least one cooling element is one of a plurality of cooling elements and said cooling elements form a mitten.

72. The cooling cover according to claim 42, wherein said at least one cooling element is one of a plurality of cooling elements and said cooling elements are provided as a stocking.

73. The cooling cover according to claim 42, further comprising one of a code and a color code.

74. The cooling cover according to claim 43, wherein said metal wool is made from a material selected from the group consisting of aluminum, copper and steel.

75. The cooling cover according to claim 44, wherein said metal foam is made from a material selected from the group consisting of aluminum, copper and steel.

76. The cooling cover according to claim 62, wherein said fixing means is a belt with a quick-lock closure.

77. The cooling cover according to claim 76, wherein said quick-lock closure is a Velcro closure.

78. The cooling cover according to claim 42, further comprising a heat-insulating layer disposed between said at least one cooling element and one of the body part and the body.

79. The cooling cover according to claim 48, further comprising an adhesive layer disposed on a side of said at least one flexible support facing the patient.

80. A device for cooling at least a part of a body of a patient, the device comprising:
at least one cooling cover containing at least one cooling element having a cooling fluid and intended for placement on the body, said cooling element being cooled to below a freezing point prior to its application, said cooling element containing a material having good thermal conductivity as compared to said cooling fluid and absorbing said cooling fluid; and
a cooling appliance for cooling said cooling cover to a temperature of below 0° C.

81. The cooling device according to claim 80, wherein said cooling appliance includes an electrically operated refrigerating unit.

82. The cooling device according to claim 81, wherein said cooling appliance includes a Peltier element.

83. The cooling device according to claim 82, wherein said cooling appliance includes a passive container with a heat insulation layer to receive said cooling cover.

84. The cooling device according to claim 83, wherein said heat insulation layer of said container includes an evacuated silicic acid.

85. The cooling device according to claim 80, wherein said cooling appliance is integrated in a patient gurney.

86. The cooling device according to claim 80, further comprising at least one temperature sensor for measuring a temperature.

87. The cooling device according to claim 86, further comprising an evaluation unit connected to said at least one temperature sensor.

88. The cooling device according to claim 87, further comprising an acoustic and/or visual output unit connected to said evaluation unit.
